(19) United States
(12) Patent Application Publication
(10) Pub. No.: US 2011/0195446 A1
(43) Pub. Date: Aug. 11, 2011

(54) METHOD AND KIT FOR SEMEN DIAGNOSIS THROUGH COLOR CHANGES IN METHYLENE BLUE AND SEMEN QUALITY EVALUATION USING SAME

(76) Inventors: Jang Hee Lee, Chungcheongnam-do (KR); Soon Hwa Baek, Chungcheongnam-do (KR); Da Ham Lee, Chungcheongnam-do (KR); Da Won Lee, Chungcheongnam-do (KR); Dal Young Ji, Gyeonggi-do (KR); Dal Young Park, Chungcheongnam-do (KR); Gowan Gook Kim, Chungcheongnam-do (KR)

(21) Appl. No.: 13/123,202

(22) PCT Filed: Oct. 7, 2009
(86) PCT No.: PCT/KR2009/005719

§ 371 Date: Apr. 7, 2011

(30) Foreign Application Priority Data
Oct. 7, 2008 (KR) 10-2008-0098136

(51) Int. Cl. C12Q 1/02; C12M 1/00

(52) U.S. Cl. 435/29; 435/288.7

(57) ABSTRACT

The present invention relates to a method and a kit for diagnosis of semen quality (motility and concentration) through observation of color change visible with the naked eye in a solution combining methylene blue and sperm. The invention comprises a method for semen diagnosis using color coding (with a standard color table) charting the various colors the semen may change into, and a diagnostic it for testing semen using said method. When combined with methylene blue, healthier and more active sperm fades the original blue color more rapidly due to respiration activity by the sperm, which allows for semen quality evaluation. The above principle are adapted to kit from inside a tube which encases a methylene blue solution into which sample semen is introduced for easy evaluation.

### TECHNICAL FIELD

The present invention relates to a diagnosis kit which makes it possible to easily evaluate the quality of semen (motility and concentration) visually with naked eyes with the aid of the changes in colors of a semen mixed with a methylene blue without using a microscope or a certain experimental tool, to and in particular to a semen diagnosis kit and a diagnosis method of an excellent semen by using a diagnosis kit which make it possible to evaluate the quality of a semen in a simple way in such a manner that a color code having a changeable color is set, and the color appearing as a result of mixing semen and methylene blue is compared with a reference color table (color codes classified with multiple concentrations) for thereby evaluating the quality of semen in simple way.

### BACKGROUND ART

The motility of sperm in semen can be easily observed in such a manner that one drop of semen is put on a slide glass and is mixed with one drop of saline solution, and the mixture is covered by a cover class and is observed by using a microscope. The whole motility of semen is roughly checked at a 100× magnitude of microscope (evaluated by the scores of 4, 3, 2, 1, 0), and ten sperms are visually checked at a 200 to 400× magnitude, and the number of the active sperms is counted, and then the number of ten sperms is counted moving slightly the slide glass, which is repeatedly performed 5 to 10 times, so it is possible to evaluate the motility by obtaining the ratio of active (moving) sperms.

The sperm before its use might survive for a few days by storing the same in a frozen state or a diluted state (usually within one week), but the sperm just before its use is subject to a microscope test in order to judge whether or not the sperm is available since the sperm might be damaged in the course of production or storage. While the above experimental test is being conducted, the semen exposed to an external environment might be degraded in its quality.

If the semen with degraded quality is directly used, a bigger problem might occur since fertilization or impregnation does not occur. So, it is needed to prevent the use of problematic sperm, which leads to enhancing the ratio of fertilization and impregnation by using the semen with better quality by simply diagnosing the quality of semen just before its use.

However the above conventional semen quality diagnosing method has a problem in storing semen after taking the same, and the method for diagnosing the quality of semen by using a microscope takes a lot of time. Furthermore, the operation of microscope needs a lot of complicated and hard work, so there are a lot of problems when collecting the semen with excellent qualities from animals.

### DISCLOSURE OF INVENTION

Accordingly, it is an object of the present invention to recognize the semen with excellent quality in such a manner that coloring stages are set by using a phenomenon that the color of methylene blue changes depending on the motility and concentration of sperms by using a reduction reaction in a mixture of semen and methylene blue, and a reference color table is made, and the phenomenon that the blue color of the semen mixed with methylene blue changes to methylene blue depending on the motility of sperm is compared with the reference color table.

It is another object of the present invention to provide a method of using the semen of good quality and a method of preventing the use of worse semen in such a manner that the quality of semen can be easily evaluated with naked eyes without using a microscope or not through an experimental analysis, and the sperm processed by sucking the sperm and methylene blue solution into a semen straw (transparent tube) is tested to evaluate its quality just before the use for external fertilization and intrauterine insemination for thereby using the semen with better quality.

According to one aspect of the present invention, there is provided a method for evaluating the quality of semen which comprises a step for mixing sperm diluted with a sperm preservation solution and a methylene blue solution; and a step for comparing a color change level of the mixed solution with a reference color table.

According to another aspect of the present invention, there is provided a semen straw for a semen quality diagnosis which comprises an upper side and a lower side which are sealed by a sealing means, the semen straw being filled with 0.1 to 0.4 of semen layer diluted with sperm preservation solution, 0.01 to 0.04 of air layer, 0.01 to 0.06 of sealing solution layer, and 0.01 to 0.04 of air layer and 0.01 to 0.04 of methylene blue solution layer, which are provided at a volume ratio (v/v).

According to a preferred embodiment of the present invention, a sealing solution belonging to the sealing layer is at least one selected from the group consisting of percoll solution, mineral oil, 2-alcohol (glycol) and 3-alcohol (glycerin).

According to further another aspect of the present invention, there is provided a method for evaluating the quality of semen which comprises a step for preparing a sealed semen straw in such a manner that 0.1 to 0.4 of semen layer diluted with sperm preservation solution, 0.01 to 0.04 of air layer, 0.01 to 0.06 of sealing solution layer, and 0.01 to 0.04 of air layer and 0.01 to 0.04 of methylene blue solution layer are inputted in the prepared straw at a volume ratio (volume/volume) in separated forms and in sequence, respectively; a step for mixing the semen layer and the methylene blue layer in the semen straw; and a step for judging the quality of semen by comparing the color change level of the mixed layer of the semen layer and the methylene layer with a reference color table.

According to still further another aspect of the present invention, there is provided a semen quality diagnosis kit which comprises a semen straw is prepared in such a manner that 0.1 to 0.4 of air layer, 0.04 to 0.4 of sperm preservation solution layer, 0.01 to 0.04 of air layer and 0.01 to 0.04 of methylene blue solution layer are inputted into the straw in sequence at a volume ration (v/v), and the remaining portions are filled with the air layer, and the semen straw is sealed; and a semen sample the quality of which is to be tested.

According to still further another aspect of the present invention, there is provided a semen quality evaluation method which comprises a step for inserting 0.04 to 0.4 of a semen sample the quality of which is to be tested into a semen straw at a volume ratio by using a semen quality diagnosis method using a semen quality diagnosis kit of claim 5; a step for resealing and swing and mixing the inserted sperm sample; and a step for performing a methylene reduction reaction.

According to still further another aspect of the present invention, here is provided a semen quality diagnosis set which comprises a semen to quality diagnosis kit of claim 5; and a sperm of the same large amount of sperm as a sample semen the quality of which is to be evaluated.

### ADVANTAGEOUS EFFECTS

In the present invention, the process that the blue color of the sperm changes to transparent color just before the mixture is classified into six steps depending on the motility and resilience of the sperm after the mixture of sperm and methylene blue solution. The standard color code (reference color table) is prepared with respect to very light blue or no-color step (available sperm), slight blue color or sky color (doubtable sperm), and blue or dark blue before mixture (unavailable sperm). The color code representing the quality of semen by the color change steps after sperm and methylene blue solution are mixed is compared with colors, so it is possible to easily evaluate the quality of semen without using a microscope or not through an experimental analysis.

### BRIEF DESCRIPTION OF THE DRAWINGS

The present invention will become better understood with reference to the accompanying drawings which are given only by way of illustration and thus are not limitative of the present invention, wherein;

FIG. 1 is a view of a color-based simple quality evaluation semen straw according to the present invention;

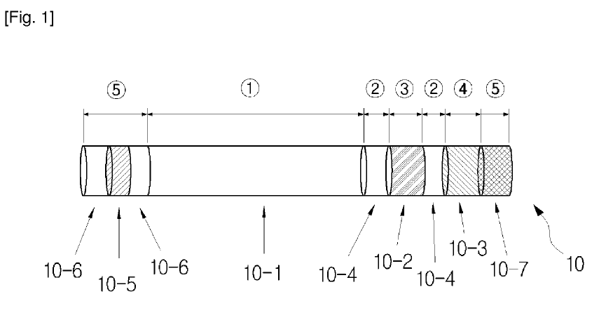

FIG. 2 is a view of a reference color table according to an embodiment of the present invention; and

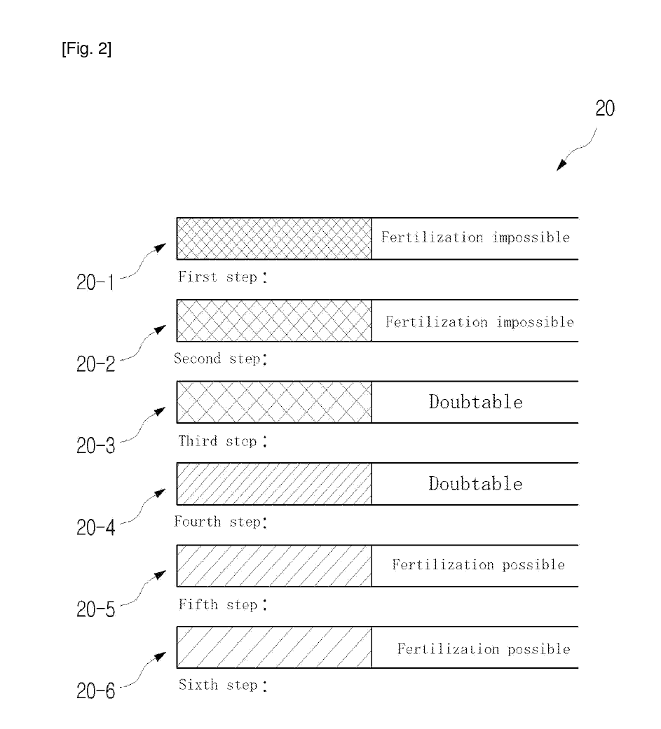

FIG. 3 is a view of a sperm diagnosis kit according to an embodiment of the present invention.

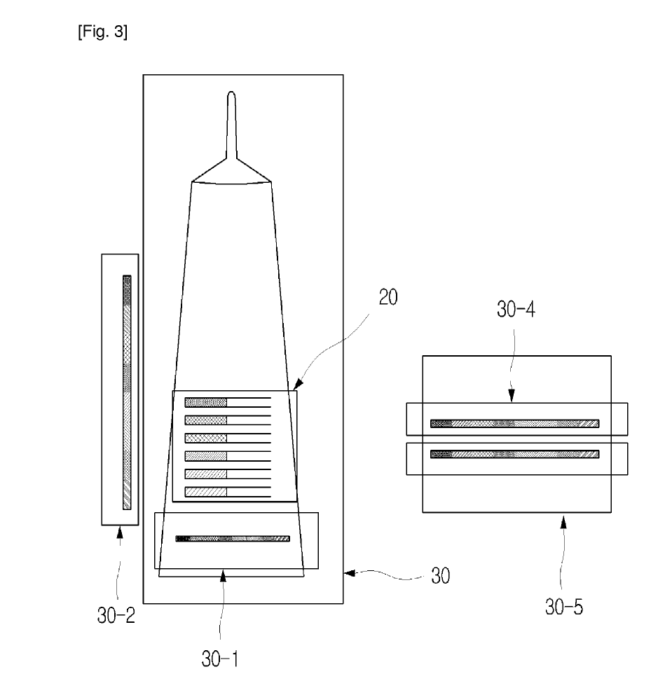

\textcircled{1} sperm diluted with sperm preservation solution
\textcircled{2} air layer
\textcircled{3} sealing solution layer \textcircled{4} methylene blue solution
\textcircled{5} semen straw upper and lower sealed layer
10: color-based simple quality evaluation semen straw
10-1: sperm diluted with sperm preservation solution
10-2: air layer
10-3: sealing solution layer (mineral oil, percoll solution, glycerin, etc)
10-4: methylene blue solution
10-5: PVA powder (sealing powder)
10-6, 7: glass ball or cotton wool
20: color comparison table with respect to methylene blue solution by which semen quality can be judged
20-1: unavailable color for use after mixing of sperm and methylene blue solution
20-2: unavailable color for use after mixing of sperm and methylene blue solution
20-3: doubtable color for use after mixing of sperm and methylene blue solution
20-4: doubtable color for use after mixing of sperm and methylene blue solution
20-5: available color for use after mixing of sperm and methylene blue solution
20-6: available color for use after mixing of sperm and methylene blue solution
30: sperm diagnosis kit
30-1, 2, 4, 5: semen straw containing sperm and methylene blue

### MODES FOR CARRYING OUT THE INVENTION

In the present invention, the sperms to be used for external fertilization and intrauterine insemination are placed in sequence in a transparent sperm tube (mini-tube: made in Germany, FHK made in Japan) (hereinafter referred to “semen straw”) in a volume-to-volume classification form of 0.1 to 0.4 of semen layer diluted with sperm preservation solution, 0.01 to 0.04 of air layer, 0.01 to 0.06 of sealing solution layer, 0.01 to 0.04 of air layer and 0.01 to 0.04 of methylene blue solution layer, respectively.

In addition, the semen straw is sealed by means of a conventional sealing member.

At this time, the mixing ratio (volume ratio) of the methylene blue solution and sperm is diluted at a ratio of 1:10 to 50 depending on the concentration of sperms. It is preferred that the concentration of diluted sperm is above 30 to 50 million sperms per ml in minimum.

When it is needed to evaluate the quality (motility and resilience) of semen just before the use of sperms, the air layer formed of double layers is touched to make it move toward upwards, which results in that the sperms and methylene solution remaining separated into air layer and sealing layer are mixed, and the sperms and the methylene blue are reduced or part (10 to 400 μl) of the sperm just before use is taken and sucked into a previously prepared sperm check kit straw and is mixed with the methylene blue, so reduction reaction occurs.

The color of the methylene blue changes from blue to light blue or transparent color (or white color) depending on the motility and resilience of sperm. In case that the motility of sperm is high, the time for changing to transparent color is short (almost within a couple of minutes), and in case that the motility is low, most of the blur color does not change, namely, it remains almost unchanged.

Since it is possible to inform the high or low state of the motility of sperm depending on the concentration of coloring of methylene blue mixed with sperm, it is possible to evaluate the motility of sperm in the semen straw by comparing with the previously prepared reference color table.

In the conventional external fertilization and intrauterine insemination, some problems might occur during the production or storage of the sperm stored in the semen straw just before the use of sperms.

Therefore, it is very anxious to check whether or not the sperm is dead, unavailable, or has very low motility and resilience enough not to be used. In order to check such problems, part of the sperm sample is taken and processed via a microscope test or other experimental processes for thereby checking the quality of semen.

However, the above tests need a lot of equipments in a laboratory room and a lot of check time, which results in very complications. Since a lot of time is needed for the above tests, the quality of semen might be degraded. Worse sperms might be actually used after the actual quality evaluation.

In the present invention, in order to prevent such problems, the sperms are classified along with methylene blue solution, intermediate air layer and sealing layer, and the air layer formed in the intermediate layer is touched by a finger, after which the sperm in the straw and the methylene solution are mixed, and the changed color is compared with the colors of the reference color tables (generally classified into six stage color codes) for thereby quickly and easily evaluating the quality of semen.

The present invention will be described in details.

In the present invention, the sperms are placed in sequence in a semen straw in a volume-to-volume classification form of 0.1 to 0.4 of semen layer diluted with sperm preservation solution, 0.01 to 0.04 of air layer, 0.01 to 0.06 of sealing solution layer, 0.01 to 0.04 of air layer and 0.01 to 0.04 of methylene blue solution layer, respectively.

The semen straw is sealed by means of a conventional sealant. Among the sealant of the semen straw, PVA has good air ventilation performance and solution hardens upon contacting for thereby obtaining a good sealing effect. The sealant might be formed of glass ball, cotton wool, synthetic fiber, PVA, plastic bar or others.

As an example of the packaging and sealing of sperm in the present invention, sperm, air, sealing solution, air and methylene blue are sucked at a proper volume ratio from the opposite opening of the straw by using a syringe connected with the silicon tube of the closed side of the semen straw (transparent plastic tube of which one side is closed after being filled with fiber layer, PVA powder layer at intermediate portion, and fiber layer, and the other side is open), and the final sperm contacts with the PVA power and is sealed, and the opposite opening is sealed by a known method. At this time, the PVA powder starts melting (dissolution) at the time when the sperm is sucked into the straw and contacts and is hardened for thereby obtaining a sealing effect.

It is obvious that no means is used so as to separate the sperm and methylene blue in the semen straw from the air and sealing solution, respectively.

The semen layer of 0.1 to 0.4, the air layer of 0.01 to 0.04, the sealing solution layer of 0.01 to 0.06, the air layer of 0.01 to 0.04, and the methylene blue solution layer of 0.01 to 0.04 diluted with the sperm preservation solution at a volume ratio (v/v) in the semen straw are classified into multiple layers and are positioned in sequence, and then the opposite opening is sealed by a known method.

The kit straw for diagnosing the quality of semen is configured like the air layer of 0.1 to 0.4, the dilution solution layer of 0.04 to 0.4, the air layer of 0.01 to 0.04, and the methylene blue solution layer of 0.01 to 0.04 are sucked at a volume ratio (v/v) by using the semen straw, and the remaining parts are classified as an air layer, and the opening used for suction is sealed by a known method. It is preferred that the kit straw is sealed by a plastic bar.

The sperm is diluted with a sperm preservation solution for obtaining a valid sperm concentration (number of sperms) after collection, the sperm preservation solution being formed of egg yolk, sodium citrate, glucose, etc. which are added as main components. Here, the sperm preservation solution means a conventional solution generally used so as to preserve sperms for a long time in living states. When it is needed to freeze sperms for a long time preservation, the dilution solution is added with glycerin (penetration type anti-freezing agent), sucrose (non-penetration type anti-freezing agent), etc., and it is sucked (or injected) into the straw of 0.25 or 0.5 ml and is sealed and cooled at 4° C. for 2 to 6 hours, and then is preliminary frozen under a liquefied nitrogen vapor environment and is dipped in liquefied nitrogen (−196° C.) and is frozen and stored. It is obvious that the kinds and spending amount of the sperm preservation solution change depending on the kinds of animals.

When an egg yolk citric acid dilution solution to be used as a sperm preservation solution is produced according to an embodiment of the present invention, 20 to 40 ml of egg yolk is added to 80 ml of 2.5 to 3.5% (weight/volume) sodium citrate. It is preferred that 20 ml of egg yolk is added to 80 ml of 2.9% sodium citrate.

When producing methylene blue solution, 30 to 60 mg of methylene blue is dissolved in 100 ml of 3.0 to 4.0% sodium citrate solution (sodium citrate 3.0 to 4.0 g/100 ml distilled water). It is preferred that 50 ml of methylene blue is dissolved in 100 ml of 3.6% sodium citrate.

It is obvious that the concentration and diluting ratio might be adjustable depending on the number of sperms (concentration) and the amount of sperm in the sperm to be mixed.

The sperm is diluted with the egg yolk citric acid. The ratio of dilution is generally 1:100 to 200 (sperm: sperm preservation solution) in case of cow, and 1:20 to 40 in case of pig, and 1:4 to 10 in case of human.

The thusly diluted sperm is sucked (injected) into the semen straw at about 0.1 to 0.4 of a volume ratio (v/v) for thereby forming a layer.

The air layer is formed in the straw at a volume ratio of 0.01 to 0.04.

Thereafter, the sealing solution is formed by sucking into a straw at a ratio of 0.01 to 0.06. The sealing solution is used to temporarily prevent the is mixture of sperm and methylene blue solution.

The sealing solution might be formed of percoll solution, mineral oil, 2-alcohol (glycol), 3-alcohol (glycerin), etc. Since sperm and methylene blue are formed of water, the sealing solution should be formed of a substance which is not well mixed with water, as a result of which two layers can be cleanly distinguished, while not largely affecting the activity of sperm and not being harmful to a biological thing.

The methylene blue layer and semen layer can be surely separated by air layer, but the air layer is movable depending on the position of straw, so it is needed for the sealing layer to separate two layers (sperm vs. methylene blue solution). It is preferred that percoll solution, mineral oil, glycerin, etc. do not affect the activity of sperm.

In addition, the air layer is formed in the straw at a volume to volume ratio of 0.01 to 0.04.

Next, the methylene blue solution is formed in the straw at a volume ratio of 0.01 to 0.04 at a volume ratio (v/v).

Finally, the straw is sealed by the known method and is stored at a is proper temperature.

When it is needed to evaluate whether or not the quality of semen is good or bad, the methylene blue solution and sealing solution are mixed with the aid of the upward and downward movements of air by mixing sperm, methylene blue solution and air, namely, by vibrating the straw a little.

The sperm in the semen mixed with the methylene blue solution emits hydrogen ion through the metabolism of sperm depending on the motility. The methylene blue reacts with hydrogen and is reduced to transparent leucomethylene blue. At this time, the colors of methylene blue change depending on the motility of sperm.

A good quality semen has a characteristic that blur color disappears within 3 to 6 minutes, and the sperm which maintains blue color for more than 7 to 9 minutes means that the number of sperms is very few or has less motility. The colors changing depending on the viability and motility of sperms are to compared with the colors of the reference color table in the above manner for thereby easily evaluating the quality of semen.

The reaction principle of the methylene blue and the sperm will be described as follows.

The sperms consume oxygen in the course of energy metabolism as a result of which hydrogen is produced. The methylene blue reacts with hydrogen and is reduced to transparent leucomethylene blue. The time needed to change to leucomethylene blue depends on the number of sperms in semen and motility. Namely, the sperms having high motility consume oxygen faster than the sperms having low motility and then produces a lot of hydrogen, so the reduction time (MRT: methylene blue reduction time) of methylene blue can be shortened.

<Principle>

2H₂O+sperm→4H⁺+O₂ (oxygen consumption during energy consumption of sperm)

Methylene blue+H⁺→Leucomethylene blue (white color)

Namely, the blur color of the methylene blue changes to to transparent color depending on the motility (when concentration exceeds a certain level) of sperm.

When the number of sperms is large and when the motility is high, MRT becomes shorter, and when the number of sperms is small, and the motility is low, the changes do not happen in colors.

The method for evaluating the quality of semen of animals based on the above methods is provided.

In the conventional transparent semen straw, 0.1 to 0.4 of semen layer diluted with sperm preservation solution, 0.01 to 0.04 of air layer, 0.01 to 0.06 of sealing solution layer, 0.01 to 0.04 of air layer and 0.01 to 0.04 of methylene blue solution layer are separately inputted in sequence and sealed by a conventional method.

The sperm for diagnosis is mixed with methylene blue and sperm, and the levels of color changes are checked after three or five minutes are passed for thereby evaluating the evaluating of sperms. In this case, the levels of color changes are visually checked with naked eyes by comparing the reference color table made by measuring standard sperms with the color changes of the sperms for diagnosis.

2. The diagnosis for easily evaluating the quality of semen of animals based on the above method is provided.

0.1 to 0.4 of air layer, 0.04 to 0.4 of diluted solution layer, 0.01 to 0.04 of air layer, and 0.01 to 0.04 of methylene solution layer are sucked into is the semen straw at a volume ratio (v/v), and the remaining portions are classified as air layer, and the opening used for suction is sealed by a conventional method for thereby manufacturing a semen quality check kit. The opening of the kit is cut (or opened), and 0.04 to 0.4 of a semen sample the quality of which is to be tested is inputted into the kit through the opening, and the kit is sealed and processed with methylene blue reaction for thereby evaluating the quality of semen. The method for evaluating the quality of semen is performed via the color comparison with the reference color table.

3. The sperm checked by the semen quality evaluation method for animal sperms is provided.

In order to obtain sperms having good qualities, part of sperm samples is collected in the conventional method and is observed by means of a microscope and then the availability of sperm is determined.

However, it is possible to instantly use the sperms after they are checked by using the sperms mixed with methylene blue in the above-described method of the present invention.

The present invention provides an easy quality evaluation (judgment) semen in which 0.1 to 0.4 of semen layer diluted with sperm preservation solution, 0.01 to 0.04 of air layer, 0.01 to 0.06 of sealing solution layer, and 0.01 to 0.04 of air layer and 0.01 to 0.04 of methylene blue solution layer are processed in the semen straw at a volume ratio (v/v).

The sperm configured to easily check the quality of semen in the above method is determined as a sample sperm and is subordinately attached to a large capacity sperm for thereby easily diagnosing the quality of semen by using the sample sperm, which results in providing a safe and reliable sperm. It is obvious that the sperms for sale are limited to animal sperms.

1. A method for evaluating the quality of semen, comprising:
a step for mixing sperm diluted with a sperm preservation solution and a methylene blue solution; and
a step for comparing a color change level of the mixed solution with a reference color table.

2. A semen straw for a semen quality diagnosis, comprising:
an upper side and a lower side which are sealed by a sealing means, said semen straw being filled with 0.1 to 0.4 of semen layer diluted with sperm preservation solution, 0.01 to 0.04 of air layer, 0.01 to 0.06 of sealing solution layer, and 0.01 to 0.04 of air layer and 0.01 to 0.04 of methylene blue solution layer, which are provided at a volume ratio (v/v).

3. A semen straw for a semen quality diagnosis according to claim 2, wherein a sealing solution belonging to the sealing layer is at least one selected from the group consisting of percoll solution, mineral oil, 2-alcohol (glycol) and 3-alcohol (glycerin).

4. A method for evaluating the quality of semen, comprising:
a step for preparing a sealed semen straw in such a manner that 0.1 to 0.4 of semen layer diluted with sperm preservation solution, 0.01 to 0.04 of air layer, 0.01 to 0.06 of sealing solution layer, and 0.01 to 0.04 of air layer and 0.01 to 0.04 of methylene blue solution layer are inputted in the prepared straw at a volume ratio (volume/volume) in separated forms and in sequence, respectively;
a step for mixing the semen layer and the methylene blue layer in the semen straw; and
a step for judging the quality of semen by comparing the color change level of the mixed layer of the semen layer and the methylene layer with a reference color table.

5. A semen quality diagnosis kit, comprising:
a semen straw prepared in such a manner that 0.1 to 0.4 of air layer, 0.04 to 0.4 of sperm preservation solution layer, 0.01 to 0.04 of air layer and 0.01 to 0.04 of methylene blue solution layer are inputted into the straw in sequence at a volume ration (v/v), and the remaining portions are filled with the air layer, and the semen straw is sealed; and
a semen sample the quality of which is to be tested.

6. A semen quality evaluation method, comprising:
a step for inserting 0.04 to 0.4 of a semen sample the quality of which is to be tested into a semen straw at a volume ratio by using a semen quality diagnosis method using a semen quality diagnosis kit of claim 5;
a step for resealing and swing and mixing the inserted sperm sample; and
a step for performing a methylene reduction reaction.

7. A semen quality diagnosis set, comprising:
a semen quality diagnosis kit of claim 5; and
a semen of the same large amount of semen as a sample semen the quality of which is to be evaluated.
